(19) United States
(12) Patent Application Publication
(10) Pub. No.: US 2018/0126111 A1
(43) Pub. Date: May 10, 2018

(54) CONTROLLED DELIVERY OF MEDICAL GASES USING DIFFUSION MEMBRANES

(71) Applicants: William Moon, Provo, UT (US); Thomas A. Tait, Box Elder, SD (US)

(72) Inventors: William Moon, Provo, UT (US); Thomas A. Tait, Box Elder, SD (US)

(73) Assignee: Nu-Med Plus, Salt Lake City, UT (US)

(21) Appl. No.: 15/817,134

(22) Filed: Nov. 17, 2017

Related U.S. Application Data
(63) Continuation-in-part of application No. 14/529,112, filed on Oct. 30, 2014.

(51) Int. Cl. A61M 16/14; A61M 16/12; A61M 16/08; A61M 16/18; A61M 16/00

(52) U.S. Cl. CPC A61M 16/142; A61M 16/125; A61M 16/08; A61M 2205/50; A61M 16/022; A61M 2202/0275; A61M 2016/0027; A61M 16/18

(57) ABSTRACT

A delivery system utilizing a diffusion membrane that regulates the concentration of medical gases that are administered to a patient. A storage vessel contains a neat or pure medical gas under pressure that is in fluid communication with a diffusion membrane. The medical gas is then dispensed at a specified rate by diffusion across a membrane into a carrier gas. The concentration is kept constant by regulating the pressure and carrier gas flow. Typical applications are for inhaled gases that are used in patient treatment and care.

### BACKGROUND OF THE INVENTION

### Field of Invention

The invention generally relates to the storage and dispensing of therapeutic levels of medical gases for inhalation by patients. Specifically this is a system that stores and delivers a specified dose of medical gas over time to a patient via inhalation.

### Description of Related Art

The administration of inhaled nitric oxide (NO) to patients is currently being investigated for its therapeutic effect. The use of NO has a vasodilatory effect on such patients and is particularly of importance in the case of newborns having persistent pulmonary hypertension. In such cases, the administration of NO has significantly increased the oxygen saturation in such infants.

The actual administration of NO is generally carried out by its introduction into the patient as a gas along with other normal inhalation gases given to breathe the patient. Such commercially available supplies are provided in cylinders under pressure and may be at pressures of about 2000 psi and consist of a mixture of NO in nitrogen with a concentration of NO of between about 800-2000 ppm. As such, therefore, some means must be used to reduce the pressure of the supply to acceptable levels for a patient and also to very precisely meter the amount of the NO and nitrogen mixture so that the desired concentration of NO is actually administered to the patient. Such administration must also be added in sympathy with the respiration pattern of the patient.

The concentration administered to a patient will vary according to the patient and the need for the therapy but will generally include concentrations at or lower than 150 ppm. There is, of course, a need for that concentration to be precisely metered to the patient since an excess of NO can be harmful to the patient. In addition, the administration must be efficient in a timely manner in that NO is oxidized in the presence of oxygen to nitrogen dioxide and which is a toxic compound. Therefore, care in its administration is paramount.

Current known methods of such administration, therefore have been limited somewhat to clinical situations where attending personnel are qualified from a technical sense to control the mixing and administration of the NO to a patient. Such methods have included the use of a forced ventilation device, such as a mechanical ventilator where a varying flow of breathing gas is delivered to the patient as well as gas blenders or proportioners that supply a continuous flow of the breathing gas to the patient to which NO has been added.

In the former case, the use of a ventilator is constrained in that the user must know the precise flow from the ventilator and then the amount of NO to be added is determined on a case-to-case and moment-to-moment basis. Furthermore, the flow profile in forced ventilation varies continuously thereby making it impossible to track the flow manually. In the use of the latter gas blenders, the introduction of the NO containing nitrogen has been accomplished through the use of hand adjustment of a gas proportioner in accordance with a monitor that reads the concentration of NO being administered to the patient. Thus the actual concentration is continuously being adjusted by the user in accordance with the ongoing conditions of the apparatus providing the breathing mixture.

While such modes of providing a known concentration of NO to the patient may be acceptable from a closely controlled and monitored clinical setting, it is advantageous to have a system that could be used with various means of providing the breathing gas, whether by mechanical means such as a ventilator, or by the use of a gas proportioner and which could automatically adjust for that particular equipment and assure the user that the desired, proper concentration of NO is being administered to the patient.

U.S. Pat. No. 5,558,083 discloses a nitric oxide delivery system that is useable with any of a variety of gas delivery systems that provide breathing gas to a patient. The system detects the flow of gas delivered from the gas delivery system at various times and calculates the flow of a stream of nitric oxide in a diluent gas from a gas control valve. The flow of gas from the gas delivery system and the flow established from the flow control valve create a mixture having the desired concentration of nitric oxide for the patient.

The system does not have to interrogate the particular gas delivery system being used but is an independent system that can be used with various flows, flow profiles and the like from gas delivery systems.

Another method of dilution consists of pre-diluting the nitric oxide with nitrogen and filling a pressurized steel tank. The drawbacks of this method is the tanks are necessarily large to provide enough gas to complete the treatment, there are multiple dilution steps necessary to create the correct dilution with more chance for errors.

The method that is described here is direct dilution method using physical constants to control the process. Direct dilution avoids potential errors. It also allows the delivery system to use a concentrated gas thereby reducing the size of the equipment and creating a potentially portable device.

### SUMMARY

A delivery system utilizing a diffusion membrane that regulates the concentration of medical gases that are administered to a patient. A storage vessel contains a neat or pure medical gas under pressure that is in fluid communication with a diffusion membrane. The medical gas is then dispensed at a specified rate by diffusion across a membrane into a carrier gas. The concentration is kept constant by regulating the pressure and carrier gas flow. Typical applications are for inhaled gases that are used in patient treatment and care.

Advantages of the system may include one or more of the following. The system provides control the administration and concentration of drugs via inhalation for the treatment of various medical conditions. In particular nitric oxide is a gas that is used in the treatment of several medical conditions. The system using a diluting gas to allow control of the nitric oxide concentrations to safe and therapeutic levels without the use of nitrogen as a diluent the concentration of the gas.

These and other features and advantages of the present invention will be more apparent from the detailed description of the preferred embodiment set forth below, taken in conjunction with the accompanying drawings.

### BRIEF DESCRIPTION OF THE DRAWINGS

FIG. 1 shows an exemplary nitric oxide delivery system.

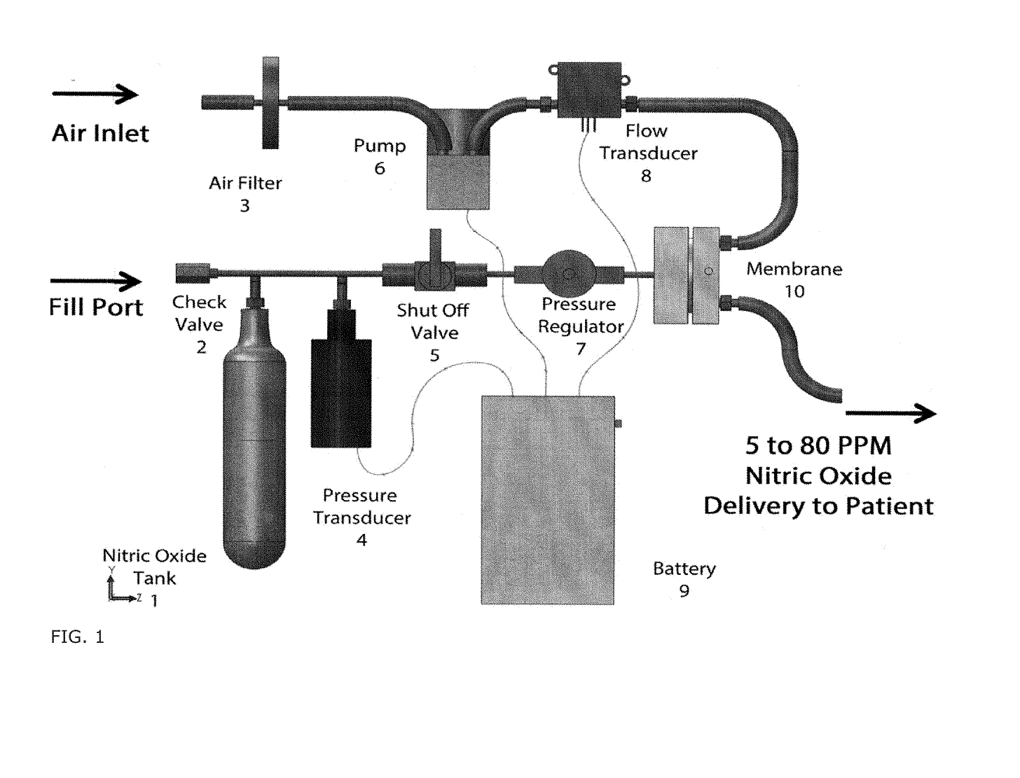

FIG. 2 shows an exemplary process for regulating and delivering the gas to the patient.

FIG. 3 shows an exemplary system to provide NO air to a patient.

### DETAILED DESCRIPTION OF THE INVENTION

The invention consists of a vessel (2) that is initially loaded with a medical gas at a pressure greater than atmospheric. The size of the vessel and the pressure control the quantity of medical gas that is available for patient treatment. As a consequence small vessels with very high pressures can contain large amounts of medical gas. The pressure is dependent on the stability of the gas under pressure. Also the gas could exist as a liquid if the temperature and pressure is below the critical point of the gas. This can be advantageous for medical gases that require a significant dose over time. A large amount of the therapeutic gas could be contained in a small vessel as a liquid that would vaporize over time.

The vessel is attached directly to a pressure regulator (6) that controls the outlet pressure of the medical gas. The pressure regulator is of the diaphragm type. The diaphragm and other materials that come in contact with the medical gas are of a material that no chemical, catalytic or absorptive interaction takes place between the material and the medical gas. The pressure range is set to a pressure that maintains the diffusion rate of the medical gas through the membrane at the desired therapeutic level. The pressure is measured by means of a pressure-sensing device (3) that measures gauge pressure. The gauge pressure is one of the control points of the system that modifies the medical gas administration rate.

The diffusion membrane (7a) is housed in a containment device (7) that supports the membrane and can withstand the pressure that is provided by the outflow from the pressure regulator. The membrane itself is comprised of a material that can withstand pressure without tearing or otherwise succumbing to mechanical stress. It also must be of a molecular structure that allows the diffusion of a selected medical gas across its thickness.

The variables involved in selecting the diffusion membrane for a specific gas are molecular composition, area and thickness. Materials that are appropriate for the diffusion membrane are typically polymers. The polymeric structure lends itself to creating diffusion pathways that are gas specific. The thicknesses of polymeric materials are easily controlled through material processing. The thickness of the polymer material is proportional to the diffusion rate as is the area. Changing the physical dimensions of the containment device can vary the area of the membrane that is exposed to the pressure of the medical gas thereby allowing more or less of the gas to reach a patient.

The physical configuration of the polymer membrane is not limited to a flat sheet. It can also be pleated, tubing, multi-lumen tubing or any physical configuration that allows a pressure differential to exist across the membrane.

Polytetrafluoroethylene (PTFE), polydimethylsiloxane (PDMS), amorphous polymers, fluorinated ethylene propylene (FEP), low-density polyethylene (LDPE) and polysilylacetalenes were excellent candidates for use in regulating the diffusion of medical gases. The selection of the membrane physical properties is driven by the usable medical gas concentration gradient that occurs across the membrane thickness. If the gradient is too steep there will be insufficient medical gas available for a therapeutic treatment. The concentration gradient is dependent upon the solubility of the medical gas in the polymer and the partial pressure on both sides of the membrane. The final selection criteria of the polymer are based on chemical stability with regard to the medical gas, adsorption and physical strength of the material. The material must be chemically stable under the pressure and temperature conditions while being intimately in contact with the medical gas. Excessive adsorption of the medical gas by the polymer can cause decreased or complete cessation of diffusion over time. The polymer membrane material must also be able to withstand the anticipated pressure and temperatures without loss of structural integrity.

The dilution gas is provided by a pump (10) or other source of dilution gas and the flow is regulated. The flow is measured by a device such as a hot wire flow transducer (9).

As an example a preferred embodiment it was experimentally determined that an optimal thickness of a polymeric structure for delivering a therapeutic dose of NO via nasal cannula was about 0.003 inches. The pure nitric oxide is loaded into a steel vessel at 120 PSI and the pressure regulated between 1 and 10 PSI. The area of the membrane was set at 1735 sq mm. With a diluent gas of air flowing at 3 liters/min the nitric oxide was able to be delivered in concentrations from 5 to 80 ppm to a patient via nasal cannula.

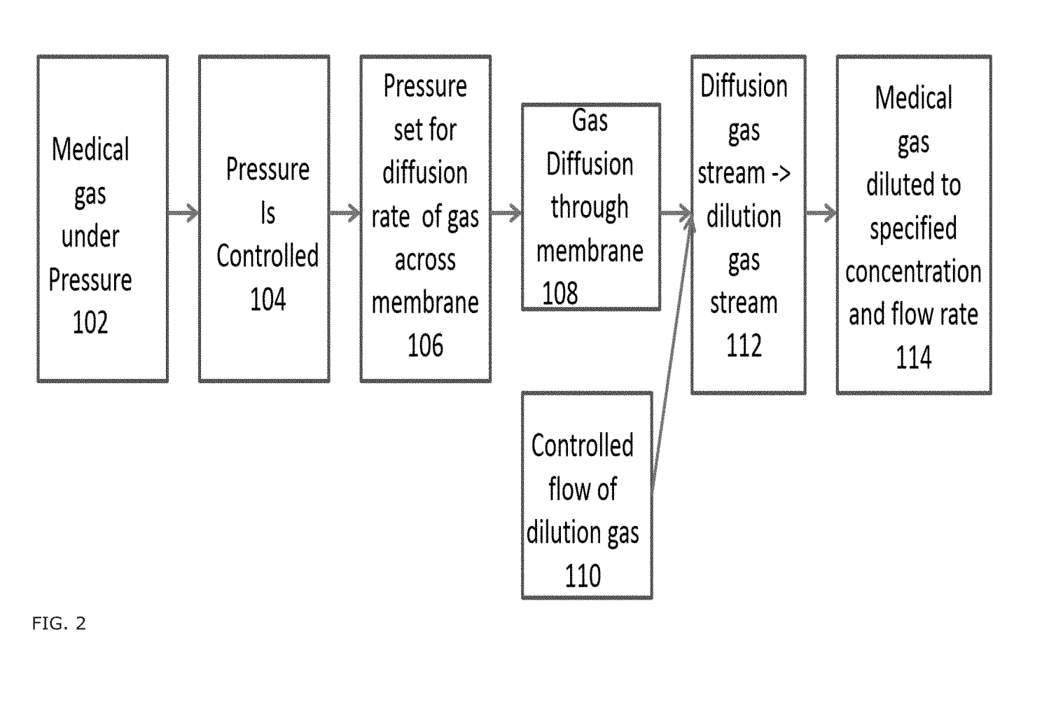

FIG. 2 shows an exemplary process for providing gas to a patient. First, the medical gas is placed under pressure (102) and the pressure is regulated (104). The user sets the rate of gas diffusion through the membrane (106). The gas is allowed to diffuse through the membrane (108), and dilution gas is also flowed through the system (110). The diffusion gas stream is provided to the dilution gas stream (112). The medical gas is diluted to the specified concentration and flow rate and delivered to the patient (114).

The existence of both a pressure control point and a flow control point allow for control scenarios that do not exist in current gas dilution/delivery products. The pressure control point using a pressure regulator provides a course control of the medical gas diffusion and ultimately concentration by increasing or decreasing the positive pressure against the diffusion membrane. The time required (seconds to minutes) for a medical gas to diffuse across the membrane with increase in pressure and the time required (minutes) for the pressure to dissipate with decrease in pressure make the pressure regulator a course control designed for large changes in gas diffusion rate. In addition, due to the nature of diffusion, it requires an intrinsic time element for the stabilization of the diffusion and thus the concentration available for precise dilution.

However once the pressure has reached a specified value and sufficient time has passed to reach a steady state the diffusion is now constant. Since the rate of diffusion of a medical gas across a membrane is now held constant by controlling the applied pressure to one side of the membrane as indicated previously an instantaneous change in concentration can be achieved by controlling the amount or flow per unit time of dilution gas provided by an air pump or pressurized gas source. Fine control is achieved by utilizing flow control either by control of the air pump flow rate or by inserting a flow control valve inline after the air pump. We can now take advantage of a fine control using time as a variable in the delivery process.

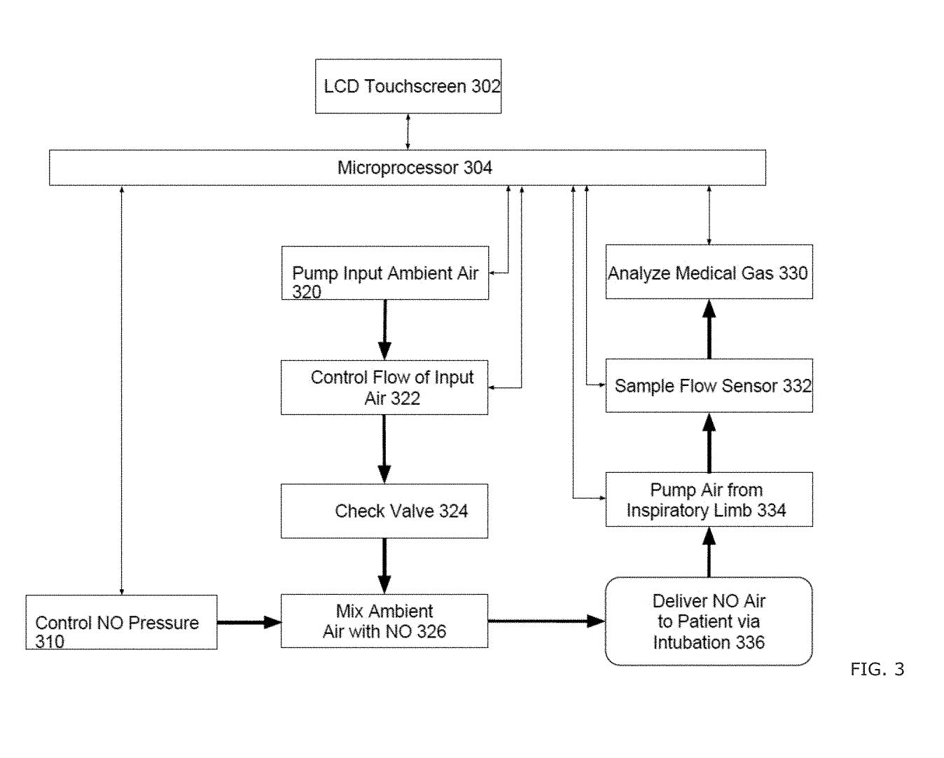

FIG. 3 shows an exemplary system to provide NO air to a patient. The system includes a touchscreen display 302 controlled by a microprocessor 304. The process receives a control input 310 on the NO pressure. The processor 304 also controls pump input ambient air 320, and input air flow is controlled at 322. The valve is checked in 324, and based on control input for the NO pressure 310, the system mixes ambient air with NO at 326. The NO air is delivered to a patient via intubation at 336. Air is pumped from the inspiratory limb 334 and the sample flow is detected by a sensor at 332. The gas is analyzed by the processor at 330. The system can utilize an electronically controlled version of a pressure regulator, a mass flow controller with a constant flow source (either air pump or compressed gas), medical gas analyzer and a microprocessor controlled feedback loop to effectively control a precise concentration of a medical gas at a very rapid rate once a concentration range for treatment has been determined. This rapid rate can be as fast as a per breath change when treatment is administered through a ventilator. This level of concentration and delivery control has not been achieved with other methodologies of dilution and delivery.

The feedback control point is placed at a point immediately prior to patient delivery. The control point consists of a sensor that is either placed in the flow path of the diluted medical gas stream or is sampled by means of a vacuum pump and delivered to a sensor that is capable of rapid detection of the specific medial gas at a concentration that is being used as a therapeutic agent. The signal from the sensor is used to determine the inspired concentration of the medical gas and by means of sending to the microprocessor where the microprocessor evaluates the signal and assigns a concentration value. This concentration value is compared to the original delivery set point concentration and if different within a specified range a signal is sent to the mass flow controller to adjust the dilution gas flow based on a lookup table of values that have been stored in the microprocessor memory (via a calibration process). The new flow rate adjusts the dilution to achieve a new corrected concentration and the process is repeated keeping the concentration within a specified concentration range as set by the user via the microprocessor. Large changes in concentration based on the medical gas being used and the therapeutic dose is achieved by utilizing the electronically actuated pressure control via an input signal from the microprocessor. The user via a user interface with the microprocessor sets this value. The system also includes various controls, alarms and safety devices to prevent excess concentrations of NO2 in the administration of NO to the patient, including means to shut down the NO system or to reduce the NO concentration to the patient to a safer level. The NO delivery system may thus provide an alarm or other appropriate action in the event of an increase in the NO level beyond a predetermined level, a decrease in O2 below a predetermined level and/or an increase of NO2 above a predetermined level. Depending on the severity of the alarm condition, an alarm may sound or the entire system may be controlled to alleviate the unsafe condition sensed.

Finally, in the event of a loss of pressure in the supply at any time, electronics can activate a purge valve to purge the system of any other gases that may be in the supply line and refill the supply lines from cylinder or tank 2 to the purge valve with fresh NO/nitrogen. In this way, the system is recharged with the correct supply gas and no extraneous gases, such as ambient air, will be introduced into the system to cause error.

Accordingly, through the use of the present NO delivery system, the concentration of NO delivered to the patient may be established, either by the selection by the user, or set by a predetermined value by the system itself, and that desired value will be transmitted to the patient without any interrogation of the gas delivery device. The system is thus independent and may be readily used with any mechanical ventilator, gas proportioning device or other gas delivery system to deliver a known, desired concentration of NO to a patient.

Numerous further variations and combinations of the features discussed above can be utilized without departing from the spirit of the invention as defined by the claims. Accordingly, the foregoing description of the preferred embodiment should be taken by way of illustration rather than by way of limitation of the invention as claimed.

What is claimed is:

1. A therapeutic level medical gas delivery system comprised of:
a pressure regulator providing a control point for the diffusion flow of medical gas through a diffusion membrane having a diffusion membrane including molecular composition in a polymeric structure having gas specific diffusion pathways to provide a predetermined diffusion flow into a carrier gas, wherein the pressure regulator keeps a concentration and flow rate of a medical gas constant by regulating pressure and a carrier gas, wherein the polymeric structure has a thickness of less than 0.05 inch to regulate nitric oxide;
a pressure-measuring device to monitor pressure; and
a containment device that allows a pressure differential to exist across a diffusion membrane such that structural integrity of the membrane is maintained.

2. A system according to claim 1, comprising a configuration unit that allows the carrier gas to dilute the pure gas diffusing from a membrane.

3. A system according to claim 1, wherein the membrane is a tube.

4. A system according to claim 1, wherein the membrane is a multi-lumen tubing configuration.

5. A system according to claim 1, comprising a vessel initially loaded with the medical gas at a pressure greater than atmospheric.

6. A system according to claim 5, wherein a size of the vessel and pressure control a quantity of medical gas available for patient treatment.

7. A system according to claim 5, wherein pressure is dependent on a stability of the gas under pressure.

8. A system according to claim 5, wherein the gas exists as a liquid if temperature and pressure are below a critical point of the gas, wherein the gas is contained in a small vessel as a liquid that vaporizes over time.

9. A system according to claim 5, wherein the vessel is attached directly to a pressure regulator that controls an outlet pressure of the medical gas.

10. A system according to claim 5, wherein the pressure regulator is a diaphragm, wherein the diaphragm and materials that contact the medical gas are of a material without chemical, catalytic or absorptive interaction.

11. A system according to claim 5, wherein the pressure range is set to a pressure that maintains a diffusion rate of the gas through the membrane at a desired therapeutic level.

12. A system according to claim 5, wherein the pressure is measured by a pressure-sensor that measures gauge pressure and modifies the medical gas administration rate.

13. A system according to claim 1, wherein the diffusion membrane is housed in a containment device that supports the membrane and withstands pressure provided by an outflow from the pressure regulator.

14. A system according to claim 1, wherein the membrane is a material that withstands pressure without tearing or otherwise succumbing to mechanical stress. It also must be of a molecular structure that allows the diffusion of a selected medical gas across its thickness.

15. A method of delivering medical gases at therapeutic levels to patients comprising:
providing a vessel that contains a medical gas at a specified pressure;
controlling the pressure for controlled diffusion across a diffusion membrane;
diffusing the medical gas across a membrane; and
maintaining concentration of medical gas through membrane composition and pressure.

16. A method according to claim 15 of introducing a carrier gas to dilute the medical gas to a therapeutic dose or concentration level.
